SEQUENCE LISTING (4 sequences)

SEQ ID NO: 1 - DNA, 18 bp, Artificial
  A synthetic PCR Primer
tcctacgggaggcagcag

SEQ ID NO: 2 - DNA, 26 bp, Artificial
  A synthetic PCR Primer
ggactaccagggtatctattcctgtt

SEQ ID NO: 3 - DNA, 25 bp, Artificial
  A synthetic PCR Primer
cgccagggttttcccagtcacgacg

SEQ ID NO: 4 - DNA, 24 bp, Artificial
  A synthetic PCR Primer
gcttccggctcgtatgttgtgtgg

(19) United States
(12) Patent Application Publication
(10) Pub. No.: US 2010/0151450 A1
(43) Pub. Date: Jun. 17, 2010

(54) Classification Method

(76) Inventor: Knut Rudi, Vestby (NO)

Correspondence Address: SCHWEGMAN, LUNDBERG & WOESSNER, P.A., P.O. BOX 2938, MINNEAPOLIS, MN, 55402, US

(21) Appl. No.: 11/922,284

(22) PCT Filed: Jun. 14, 2006
(86) PCT No.: PCT/GB2006/002169

§ 371 Date: Dec. 14, 2009

(30) Foreign Application Priority Data
Jun. 14, 2005 (GB) 0512116.5

(51) Int. Cl. C12Q 1/68

(52) U.S. Cl. 435/6

(57) ABSTRACT

The invention provides a method of classifying a microorganism present in a sample comprising the steps of (a) digesting nucleic acid derived from the microorganism with at least one restriction enzyme; and (b) determining the melting profile of the restriction fragments produced in step (a). The melting profiles may he subjected to further statistical analysis. Classification may be effected by reference to predetermined melting profiles or the results of previously analysed melting profiles. According to the invention, the melting profiles and statistical analyses obtained from the methods of the invention may be stored on digital media to produce a database. These databases and retrieval of data therefrom as part of the methods of the invention are encompassed by the present invention. Methods applicable to higher eukaryotes and kits for carrying out the methods of the invention are also provided.

The present invention relates to an explorative screening method for the identification and classification of microorganisms and other cells in a sample.

Our general knowledge about microbial communities is still relatively limited (Pace, N. R. 1997. Science 276:734-740, 7, Venter, J. C., et al 2004. Science 304:66-74.). One of the major limiting factors is the type of method used for gaining information about the communities (Theron, J., and T. E. Cloete. 2000. Crit Rev Microbiol 26:37-57.). What is still lacking are explorative screening methods to analyse large sample sets. Analyses of large sets of communities are necessary both for generalization of observations and to span the diversity of microorganisms in a given habitat (Amann, R. I., et al 1995. Microbiol Rev 59:143-169.). Explorative screenings may also be used to identify samples with divergent microbial communities that need further characterization.

Sequencing 16S rDNA is considered the most accurate method for identifying and classifying bacteria and other microorganisms (Venter, ibid). DNA sequencing, however, is relatively complicated and expensive, and is certainly not suitable for routine applications in industries such as the food industry. Currently, the most widely used explorative methods to describe microbial communities are rDNA restriction fragment length polymorphism (tRFLP), temperature/denaturing gradient gel electrophoresis (TGGE/DGGE), analyses of clone libraries, or density gradient centrifugation (Acinas, S. G., et al 2004. Nature 430:551-554. Fukushima, H., et al. 2003. J Clin Microbiol 41:5134-5146. Domann, E., G. et al 2003. J Clin Microbiol 41:5500-5510. Muyzer, G., and K. Smalla. 1998. Antonie Van Leeuwenhoek 73:127-141). Common to these explorative methods is that they are based on the physical separation of DNA fragments. Methods based on physical separation, however, are relatively complicated and cannot easily be adapted for high-throughput applications.

The most widely used methods for microbial classification in the food industry are based on phenotypic characteristics such as sugar fermentation patterns. Determining sugar fermentation patterns, however, is relatively laborious and time-consuming. Recently, more rapid spectroscopic techniques such as FT-IR have been developed for determination of microbial phenotypes (Orsini, F. D. et al 2000. J Microbiol Methods 42:17-27). The limitation with spectroscopic techniques, however, is difficulties with standardization since these techniques require highly defined microbial growth conditions.

Thus, there is the need for a microbial classification technique which is simple, fast, cost effective, and capable of adaptation to high throughput screening protocols.

The present invention addresses these problems. The inventors have for the first time recognised that different microorganisms have characteristic restriction fragment melting curve signatures. Restriction enzymes are enzymes that cleave nucleic acids at specific sites in regions of specific nucleotide sequence, so called restriction sites. The resulting fragments are restriction fragments. Double stranded nucleic acid melts into single strands when heated sufficiently. The temperature at which melting occurs depends on the length and the nucleotide sequence of the nucleic acid. Because different microorganisms have different genetic sequences the pattern of restriction sites differs and therefore the array of fragments that are generated by a restriction enzyme will differ. Every fragment will have a different size and/or sequence and so will have a different melting curve. Each fragment's melting curve contributes to an overall restriction fragment melting profile for the microorganism. Different microorganisms will have different restriction fragment melting profiles as a result of differences in the genetic code of the microorganism. These profiles can be thought of as characteristic restriction fragment melting curve signatures.

These differences form the basis of the present invention. The basic idea of restriction fragment melting curve analysis (RFMCA) is to use differences in restriction fragment melting curves rather than physical separation on the basis of size to analyse patterns of restriction enzyme cut DNA from complex samples. One benefit of RFMCA is that the whole analysis can be done in a single tube and thus the approach is suitable for high-throughput protocols. RFMCA is also explorative, unlike other real-time melting point assays, which are designed for detecting only specifically targeted bacteria or bacterial groups (Fukushima, H. et al 2003, J. Clin. Microbiol 41: 5134-5146) or specific single nucleotide polymorphisms (SNP's) in eukaryotes (Ye, J., et al., J. Forensic Sci., 2002; 47(3): 593-600). The latter two approaches rely on predetermined polymorphisms and/or known fragment sizes to enable detection whereas the present invention utilises the unique melting curves which arise from polymorphisms, which may be unknown, that are specific to a particular microorganism.

Thus, in a first aspect there is provided a method of classifying a microorganism present in a sample comprising the steps of:

a) digesting nucleic acid derived from the microorganism with at least one restriction enzyme; and

b) determining the melting profile of the restriction fragments produced in step a).

Preferably an initial step is performed wherein a target region in the nucleic acid of the microorganism is amplified. In this case the digestion will be performed on the amplification products of the initial step and the digested nucleic acid will be ‘derived from’ the microorganism in that sense. Thus preferably the nucleic acid derived from the microorganism will be nucleic acid obtained through amplification of a target region of the nucleic acid of the microorganism.

By “microorganism” it is meant organisms that are of the microscopic scale. Typically such organisms will be unicellular. Non-limiting examples include bacteria, fungi, the protists, algae, protozoa, viruses and mycoplasma. The method of the invention is particularly suited to the classification of bacteria. Table 1 provides examples of the bacteria that may be classified using the method of the invention.

The method of the invention is applicable to complex samples of microorganisms and is capable of classifying a plurality of different types of microorganisms in a single sample without the need for separation and/or separate culture prior to classification. Thus, 2 or more, 3 or more, 5 or more even 8 or more different microorganisms in a sample may be classified simultaneously.

Preferably the method of the invention can classify microorganisms in a sample at least to the level their taxonomic family, more preferably at least to the level of their taxonomic genus, and most preferably at least to the level of their taxonomic species.

“Taxonomic family” is defined as a taxonomic category of higher rank (i.e. more inclusive) than genus but of lower rank (i.e. less inclusive) than order. Non-limiting examples include Enterobacteriaceae, Pasteurellaceae, Mycoplasmataceae, Pseudomonadaceae, Chromatiaceae, Micrococcaceae, Methanobacteriaceae.

“Taxonomic genus” is defined as a taxonomic category of higher rank (i.e. more inclusive) than species but of lower rank (i.e. less inclusive) than family. Non-limiting examples include Escherichia, Salmonella, Staphylococcus, Listeria, Bacillus, Hyphomicrobium, Entamoeba, Toxoplasma, Giardia, Rhizopus, Blastomyces and Saccharomyces.

“Taxonomic species” is defined as a taxonomic category of higher rank (i.e. more inclusive) than subspecies but of lower rank (i.e. less inclusive) than genus. Non-limiting examples include Escherichia coli, Salmonella typhi, Staphylococcus aureus, Listeria monocytogenes, Bacillus subtillis, Entamoeba histolytica, Rhizopus stolonifer, Blastomyces dermatitidis, Saccharomyces cerevisiae. Further examples are provided in Table 1.

Classification of microorganisms to these taxonomic levels might, however, not be required in some instances. Classification may merely be in terms of confirming that a sample of microorganisms, or a microorganism, has the same restriction fragment melting profile as another sample, or microorganism. In these instances a taxonomic label might not be assigned at all.

The taxonomic level to which a microorganism can be classified with the method of the invention may be dependent on the target region amplified. The target region should preferably be a region of nucleic acid in which evolutionary differences between different taxonomic families/genera/species are present in the sequence of the target region. The level of resolution required will dictate the choice of target region. For instance, if the target region is 16S rDNA different microorganisms can be classified to the genus level. If the spacer between 16S rDNA and 23S rDNA is the target region microorganisms can be classified to the species level. These two are preferred target regions. The skilled man can therefore select a suitable target region depending on the degree of resolution required, the nature and diversity of microorganisms present in the sample etc. Further examples of suitable sequences include, but are not limited to, 23S rDNA and genomic sequences encoding nucleic acid elongation factors, ATPases and other housekeeping genes. The type of nucleic acid that can be used is not important. Therefore DNA, RNA, PNA and single, double or multi strand forms thereof may be used so long as the requisite evolutionary differences in the sequence exist.

The nucleic acid which undergoes amplification according to the method of the present invention is typically obtained from the microorganisms in the sample in any standard way. From his common general knowledge the skilled person will be capable of obtaining nucleic acid of sufficient quality and quantity to allow amplification. The choice of extraction technique will depend on the sample which contains the microorganisms to be classified. Samples from which microorganisms are classified according to the invention include environmental samples such as water samples, e.g. from lakes, rivers, sewage plants and other water-treatment centres or soil samples. The methods are of particular utility in the analysis of food samples and generally in health and hygiene applications where it is desired to monitor microorganism levels and/or identity, e.g. in areas where food is being prepared. Milk products for example may be analysed for listeria. Food such as cheese, ice cream, eggs, margarine, fish, shrimps, chicken, beef, pork ribs, wheat flour, rolled oats, boiled rice, pepper, vegetables such as tomato, broccoli, beans, peanuts and marzipan may also be analysed.

Samples from which microorganisms may be classified according to the present method may be clinical samples taken from the human or animal body. Suitable samples include, whole blood and blood derived products, urine, faeces, cerebrospinal fluid or any other body fluids as well as tissue samples and samples obtained by e.g. a swab of a body cavity.

The sample may also include relatively pure or partially purified starting materials, such as semi-pure preparations obtained by cell separation processes.

Amplification of the target region can be achieved in any appropriate way. The skilled man would be readily aware of appropriate techniques. PCR will commonly be used. However alternative techniques are equally applicable. If necessary for the amplification technique chosen, the skilled man will also be able to design suitable oligonucelotide primers making use of publicly available sequence databases.

The evolutionary differences in the sequence of the target region between families/genera/species affect the frequency at which any particular restriction enzyme cuts the target sequence (and therefore amplification products). As a result differences in the size of restriction fragments are observed between families/genera/species. Different sized fragments melt with different curves and so differences in the melting curves of amplification products are observed between families/genera/species. It is these differences that enable microorganisms in a sample to be distinguished and can result in classification of the family/genus/species.

These different melting point curves for the different fragments in the sample together provide an overall profile for the sample as a whole and it is this profile which is analysed to give the desired classification information. Conveniently the profile is compared with reference profiles from known samples and can be categorised as the same or similar to a known type or grouping of microorganisms to provide information about the sample under investigation. This can be basic information sufficient to confirm a microorganism is common to two or more samples. In this instance the microorganisms are classified in terms of their melting profiles but a taxonomic label is not necessarily assigned. The methods of the invention do however have sufficient resolution such that specific microorganisms in the sample can be classified to the taxonomic level of family/genus/species etc.

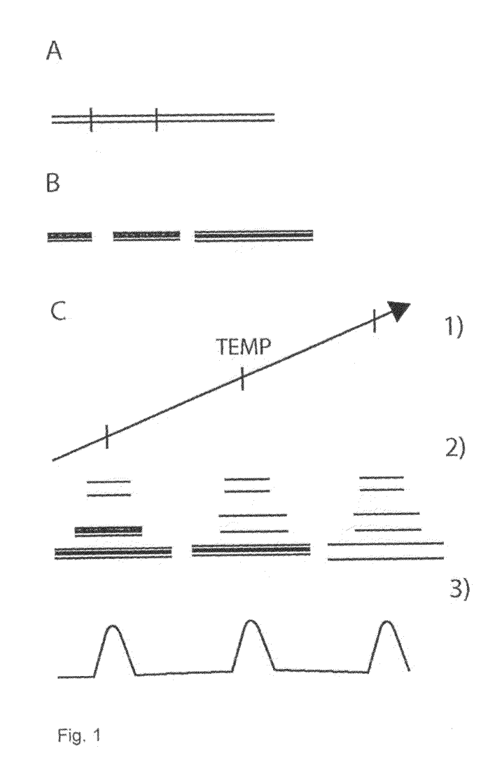

To obtain resolution between the melting curves of fragments from different families/genera/species the size of the restriction fragments can be optimised. The skilled man is able to calculate theoretical cutting frequencies for particular restriction enzymes and thus he will be able to devise suitable combinations of restriction enzymes to obtain an optimum fragment size. The general rule is that if the fragment is too large the fragment will not melt sufficiently thus impairing resolution and if the fragment is too small there will be no difference between the melting points thus also impairing resolution. The optimum size will vary as a function the taxonomic level at which classification is desired and the degree of sequence variation between the sequence of the target region. Thus, if the target region varies greatly but classification is only required to the level of family, fine resolution (and therefore a high degree of optimisation of fragment size) is not necessarily required as the differences in melting point between orders are likely to be great. On the other hand if different species are to be classified the requisite resolution is much higher and so the need for optimisation is much greater. In order to resolve two distinct peaks the minimum difference in melting points is 2.5° C. Resolution of melting points is also affected by the range at which melting occurs. As a general rule the range 65-92° C. (see FIG. 1A for typical pattern) is most suitable. The melting patterns obtained below 65° C. were relatively unstable, possibly due to variable accumulation of small fragments such as primer dimers. All the fragments were melted above 92° C., and thus no useful information was obtained above that temperature.

Preferably more than one different restriction enzyme is used, more preferably at least two most preferably at least 3 or 4.

In order to achieve a signature profile which can be used to obtain useful classification information a minimum number of obtained fragments after restriction digestion is desirable, preferably at least 5 different fragments, more preferably at least 8 or 10, most preferably at least 12 or 15 different fragments, e.g. 10-20 or 10-30 different fragments.

For any target region the fragment length should be between 300 and 30 bp, preferably between 200 and 40 bp and most preferably between 100 and 50 bp. These ranges provide distinct melting points in the range 65-92° C. Examples of restriction enzymes that produce 256 bp fragments of 16S rDNA when used singularly and 64 by fragments when used in combination are MspI (C▾CGG), AluI, (AG▾CT), MseI (T▾TAA) and RsaI (GT▾AC). Combinations of these enzymes constitute preferred embodiments of the present invention.

A further parameter that may be optimised is the stringency of the buffer in which the melting reaction is performed. The skilled man will be aware of agents that would affect the stringency of the melting buffer. By way of example, high salt standard saline citrate (SSC) solution would lower the stringency and dimethylsulfoxide (DMSO) would increase the stringency.

Measurement of restriction fragment melting profiles can be performed in any appropriate way. The skilled man would be aware of such techniques. Measurement of melting curves may conveniently be performed in any commercial Real Time PCR apparatus, examples of which include the ABI Prism 7700 Sequence Detection System or the 7900HT system (Applied Biosystems). Dissociation Curves 1.0 software (Applied Biosystems) can be used to analyse the melting patterns for the 7700 data, while SDS 2.2 software (Applied Biosystems) can be used to analyse the data generated with the 7900 HT system.

Raw data obtained from the melting reaction may be used to classify the microorganisms present in a sample. Comparison of the melting profiles with reference profiles from known microorganisms is sufficient to make the classification. The reference profile need only be determined once for a particular target region of a particular microorganism, data obtained from later samples need only be compared with the reference profile to make the classification. Typically, a pure sample of a particular microorganism will be used to obtain the reference profile. A database of melting profiles can therefore be maintained and the melting profiles for each new sample need only be compared with the database to effect the classification.

Classification models may also be generated from the melting curve data using bilinear modelling methods such as principal component analyses (PCA) or multivariate regression methods such as partial least square regression (PLSR) in combination with the prediction tools provided in the Unscrambler software (Camo Inc, Woodbridge, N.J.) or any other software suitable for performing multivariate statistical analyses. The results of these analyses enables the user to assign a test microorganism in a sample to a predetermined classification grouping. This grouping and the microorganisms contained therein must be predetermined. This is preferably by clustering RFMCA data around phylogenetic trees which have been predetermined using data obtained from sequencing based techniques. This clustering is conveniently achieved using correlation coefficient distances and Ward linkage for dendrogram construction although other techniques can be employed.

For a particular microorganism and a particular target region the original clustering need only be made once. Typically, a pure sample of a particular microorganism will be used to obtain the reference clustering information. A database of clustering information can therefore be maintained and the statistical results for each new sample need only be compared with the database to effect the classification.

It will be appreciated that a database of melting profiles and/or clustering information may be in any computer readable form, for example as data in a relational database such as Microsoft Office Access™, Oracle® and so forth, or data in a spreadsheet for example. The database may be supplied on a stand-alone basis or on a network, hosted on a server, such as on a corporate network or on a web server accessible over the internet. Data for creating or updating the database may be provided on physical media such as a disk, or may be provided in downloadable form from a remote location.

Where the composition of complex microorganism communities in a sample is to be assessed the use of statistical modelling techniques is normally required. The Examples provide guidance on the formulation of reference groupings and their use to allow classification of microorganism in a sample.

Phylogenetic reconstruction uses genetic distances to reconstruct evolutionary trees. The evolutionary distance between a pair of sequences usually is measured by the number of nucleotide substitutions occurring between them. There is a wide variety of options for tree constructions, ranging from simple dendrograms to more complicated methods such as neighbour-joining (NJ). NJ is a simplified version of the minimum evolution (ME) method, which uses distance measures to correct for multiple evolutionary hits at the same sites and chooses a topology showing the smallest value of the sum of all branches as an estimate of the correct tree. However, the construction of an ME tree is time-consuming because, in principle, the S values for all topologies have to be evaluated and the number of possible topologies (unrooted trees) rapidly increases with the number of taxa. In ME the sum, S, of all branch length estimates is computed for all plausible topologies, and the topology that has the smallest S value is chosen as the best tree. With the NJ method, the S value is not computed for all or many topologies. The examination of different topologies is imbedded in the algorithm and so only one tree is finally produced. This method does not require the assumption of a constant rate of evolution so it produces an unrooted tree.

RFMCA does not involve electrophoresis in order to determine fragment size. The fact that a gel-free method is provided is a preferred feature. In fact, the amplification step, the restriction step and melting reaction can be performed in the same vessel. This makes RFMCA eminently suitable for adaptation to high throughput screening protocols, to automation and to the provision of quick simple methods.

Preferably steps a) and b) are performed in the same vessel, more preferably the amplification step is also performed in that vessel.

Viewed alternatively, the invention provides a method of determining the identity of a microorganism in a sample comprising the steps of:

a) digesting nucleic acid derived from the microorganism with at least one restriction enzyme; and

b) determining the melting profile of the restriction fragments produced in step a).

By “determining the identity” it is meant assigning the microorganism that is present in a sample to a taxonomic family, preferably a taxonomic genus and most preferably to a species. The meaning of these taxonomic groupings is defined above

The invention, in a further aspect, provides a method of classifying a cell from a higher eukaryote present in a sample comprising the steps of:

a) digesting nucleic acid derived from the microorganism with at least one restriction enzyme; and

b) determining the melting profile of the restriction fragments produced in step a).

All preceding discussion in relation to the first aspect of the invention applies mutatis mutandis to this aspect of the invention.

By higher eukaryote it is meant any multicellular organism classified in the taxonomic domain Eukaryota, or alternatively, any multicellular organism from the taxonomic kingdoms Animalia, Plantae and Fungi. It is envisaged that the method of the invention can classify a cell from a higher eukaryote at least to the level their taxonomic family, preferably at least to the level their taxonomic genus, and most preferably to the level at least their taxonomic species.

“Taxonomic family” is defined as a taxonomic category of higher rank (i.e. more inclusive) than genus but of lower rank (i.e. less inclusive than order). Non-limiting examples include Felidae, Canidae, Ursidae, Poaceae, Hominidae, Brassicaceae, Drosophilidae, Cyprinidae; Muridae

“Taxonomic genus” is defined as a taxonomic category of higher rank (i.e. more inclusive) than species but of lower rank (i.e. less inclusive than family). Non-limiting examples include Felis, Panthera, Canis, Ursus, Zea, Homo, Arabidopsis, Drosophila, Dank, Rattus.

“Taxonomic species” is defined as a taxonomic category of higher rank (i.e. more inclusive) than subspecies but of lower rank (i.e. less inclusive than genus). Non-limiting examples include Felis catus, Panthera pardus, Canis familiaris, Ursus horribilus, Zea mays, Homo sapiens, Arabidopsis thaliana, Drosophila melanogaster, Danio rerio, Rattus norvegicus.

In a further aspect the present invention provides a kit for use in a classification method of the invention as defined herein, said kit comprising one or more restriction enzymes, optionally one or more primers suitable for performing an amplification reaction, optionally a restriction buffer, optionally a melting buffer, optionally means for providing an indication of nucleic acid duplex dissociation, i.e. melting of nucleic acid. This means will typically comprise a fluorescent molecule whose level or type of fluorescence alters when the nucleic acid molecule in which it is associated melts, e.g. SYBR® Green I stain.

The invention will be further described with reference to the following non-limiting Examples in which:

FIG. 1 shows the RFMCA principle. (A) The template for RFMCA is PCR amplified dsDNA. (B) This DNA is cut by restriction enzymes and stained with SYBR Green I. (C) Finally, the fragments are melted by gradual increase in the temperature (1), and the transformation from dsDNA to ssDNA (2) is recorded as a melting curve (3).

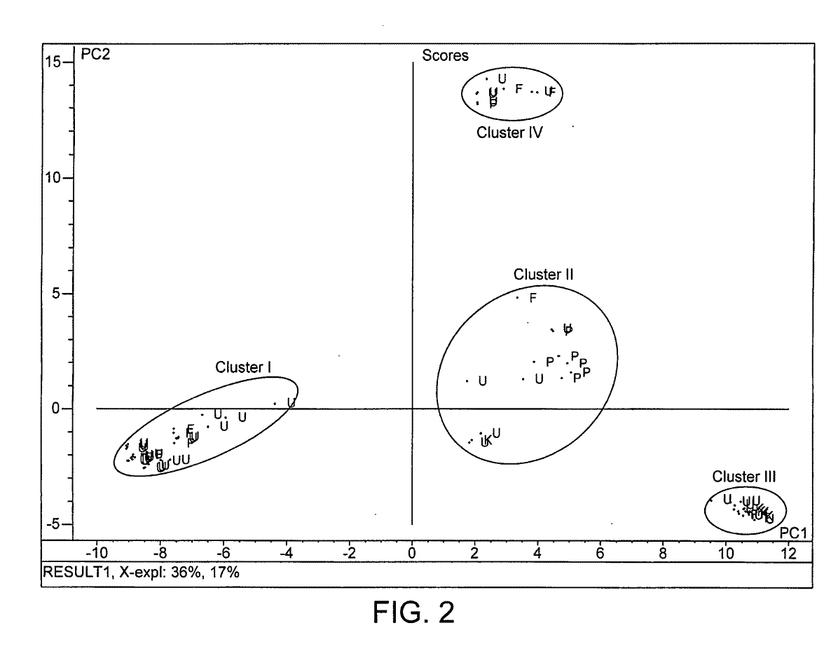

FIG. 2 shows PCA analyses of 16S rDNA sequence data. PCA analyses were performed on 72 of the strains shown in Table 1. Cluster Ito IV are marked. The following symbols were used to indicate the origin of the strains; P—pepper, K—curry chicken, F—finnbeef and U—herb sauce.

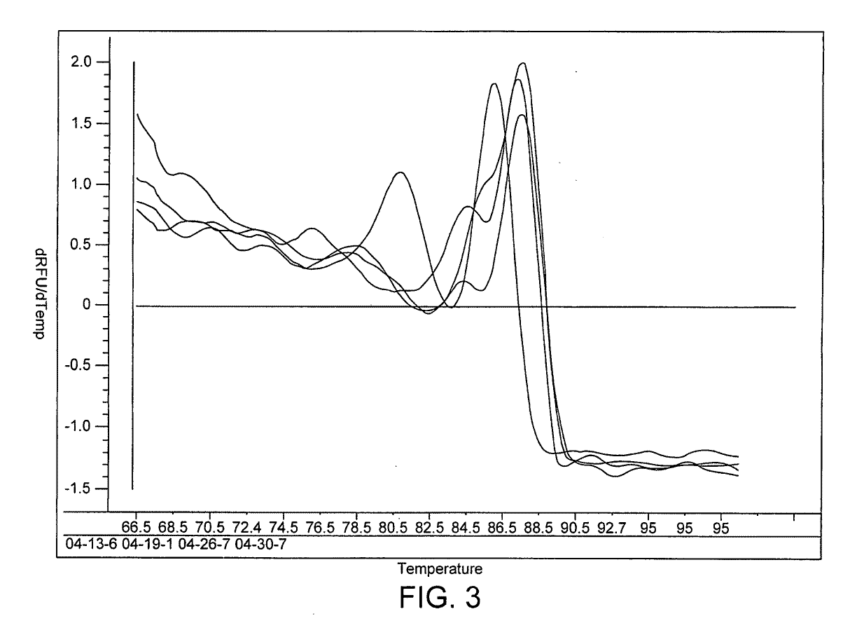

FIG. 3 shows an example of RFMCA patterns in terms of the derivative of the fluorescence change. The representative strains are 04-13-6, and 04-30-7 04-26-704-19-1 for Cluster I to IV, respectively.

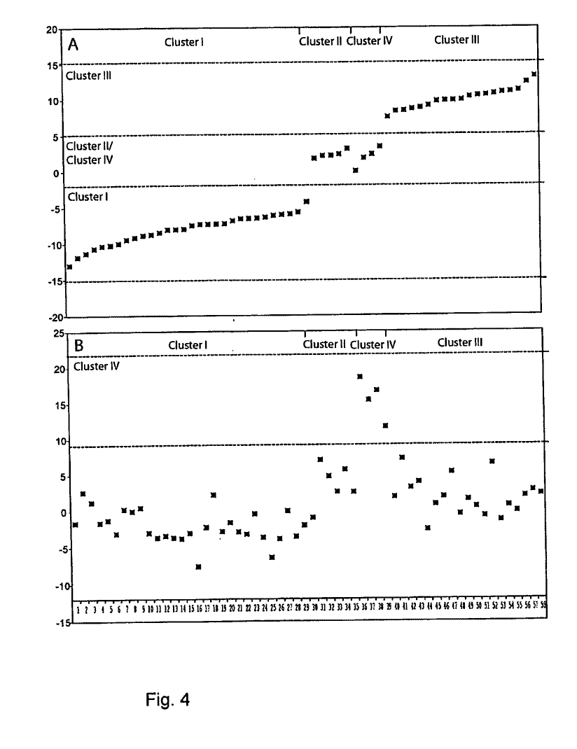

FIG. 4 shows RFMCA classification. The RFMCA classification was done based on a regression model with DNA sequence data as Y and RFMCA patterns as X. The predicted values for PC 1 (A) and PC 2 (B) are shown. The stippled lines show the cut-off values between Cluster Ito IV. The following strains were analysed; a04-10-1, a04-11-1, a04-11-3, a04-11-4, a04-11-5, a04-11-5, a04-12-1, a04-12-2, a04-12-3, a04-12-4, a04-13-1, a04-13-2, a04-13-5, a04-13-5, a04-13-6, a04-13-6, a04-13-7, a04-13-8, a04-15-1, a04-17-1, a04-17-10, a04-17-11, a04-17-12, a04-17-13, a04-17-2, a04-17-3, a04-17-4, a04-17-5, a04-17-6, a04-17-7, a04-17-8a, a04-17-8b, a04-17-9, a04-18-1, a04-19-1, a04-20-1, a04-20-1, a04-20-2, a04-20-2, a04-20-3, a04-20-3, a04-20-4, a04-20-4, a04-21-2, a04-26-7, a04-28-2, a04-28-3, a04-28-4, a04-28-5, a04-28-8, a04-29-1, a04-30-7, a04-31-1, a04-31-3, a04-31-4, a04-32-5, a04-32-7 and a04-32-8. These strains have been isolated from heat treated food.

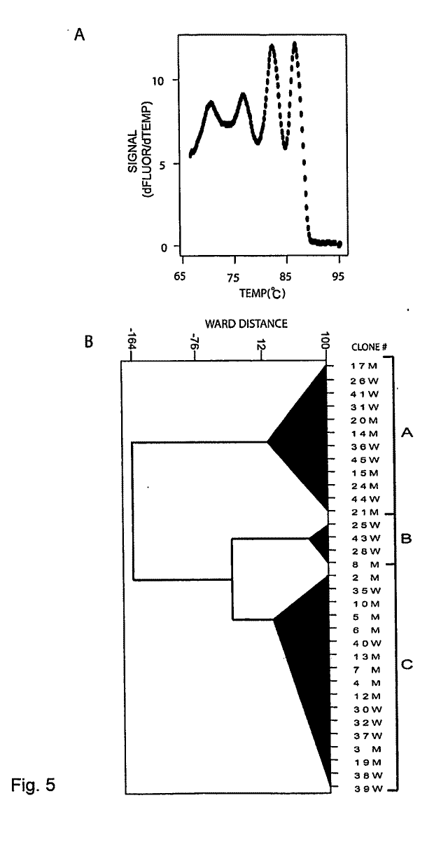

FIG. 5 shows cluster analyses for the RFMCA patterns for cloned 16S rDNA sequences. (A) The RFMCA pattern for clone 17M is shown as an example of the data used for cluster analyses. Abbreviations: dFLUOR/dTEMP, change in fluorescence signal relative to temperature. (B) The clustering was done using the Ward algorithm for linkage and correlation distances measures. The CLONE # indicates from which sample the clone was obtained.

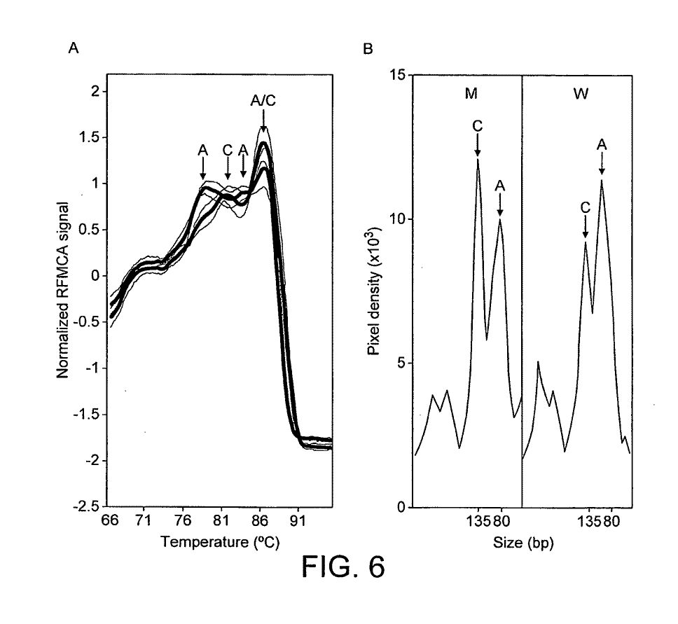

FIG. 6 shows RFMCA (A) and tRFLP (B) for the W and M samples. (A) RFMCA melting pattern for the W (dark line) and the M (light line) samples. The thin lines represent the standard deviation (eight samples for both M and W). The peaks for bacteria belonging to the A and C groups are marked with arrows. (B) The tRFLP results (TAMRA labelled reverse primer) for the W and the M samples are shown. The two main discriminatory bands for the A and C groups are marked. Abbreviations: bp, base pairs.

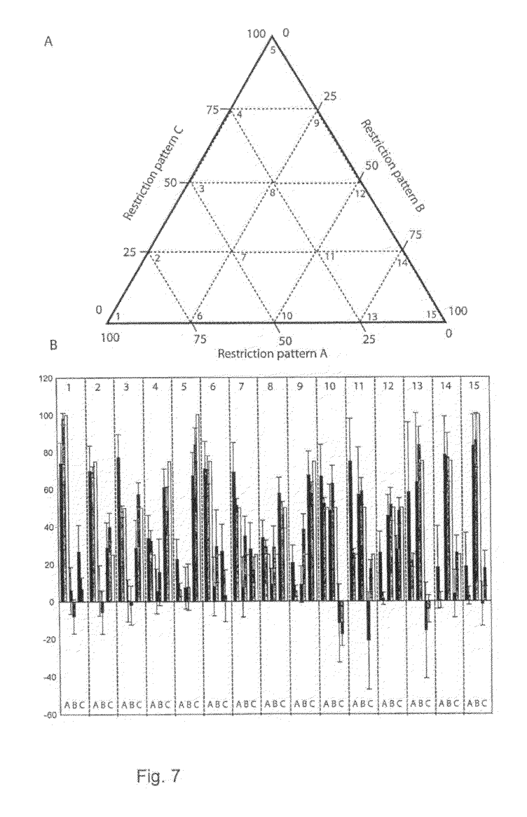

FIG. 7 shows a comparison of RFMCA and tRFLP for mixes of known components. (A) Clones with restriction patterns corresponding to the major groups of patterns A (17M), B (43W) and C (13M) identified in FIG. 1 were mixed following the experimental design shown. The numbers within the triangle indicate the numbering of the samples. (B) Predictions for the validation set of samples of the tRFLP (dark grey bars) and the RFMCA (grey bars) data for the restriction patterns A, B and C. The light grey bars show the expected values. The numbering corresponds to the numbers in panel A. The standard deviations are determined from jack-knife cross-validation.

### EXAMPLE 1

Bacterial Strains

The bacterial strains (shown in Table 1) were isolated from heat-treated food products. The bacteria were grown on standard blood agar plates (Oxoid).

PCR Amplification

DNA was purified using PrepMan Ultra following the manufacturers recommendations. PCR amplification of the purified DNA was performed using the primers 5′TCC TAC GGG AGG CAG CAG T3′ (forward) and 5′GGA CTA CCA GGG TAT CTA TTC CTG TT3′ (reverse). The primers target generally conserved regions of the 16S rRNA gene. Two μl template was used in 25 μl amplification reactions. The reactions contained 1× AmpliTaq Gold reaction buffer, 1 mM MgCl2 1 mM dNTP's, 1 μM of each primer and 1 U AmpliTaq Gold DNA polymerase. The amplification profile used was as follows: (95° C. for 30 s, 65° C. for 30 s and 72° C. for 45 s)×35. The enzyme was activated and target DNA denatured at 10 min for 95° C. prior to amplification, and an extension step of 7 min at 72° C. was included after the amplification. The reactions were performed using a GeneAmp PCR System 9700 (Applied Biosystems).

DNA Sequencing

The presequencing reaction included treating 8 μl of the PCR product with 10 U exonuclease I (Amersham, Piscataway, N.J.) and 2 U shrimp alkaline phosphatase (Amersham) at 37° C. for 15 min. The enzymes were inactivated by heating to 80° C. for 15 min. Sequencing was performed using the Big Dye™ Terminator v 2.0 Cycle Sequencing Kit (Applied Biosystems, Foster City, Calif.) on a 3100 DNA sequencer. Preparation of the sequencing mixture was performed as recommended by the manufacturer (Applied Biosystems).

Phylogenetic Reconstruction

Alignment independent bi-linear multivariate modelling (AIBIMM) was used for phylogenetic analysis. The sequences were transformed into multimer frequencies (n=6) by a C# script. The multimer frequencies were subsequently used for multivariate statistical analysis. The multimer frequency data were centred and normalized by dividing each variable by its standard deviation prior to the PCA analysis in AIBIMM. In this way, the different pentamer frequency variables have the same influence on the PCA solution regardless of the original variable variance. The NIPALS algorithm was used for PCA as implemented in the Unscrambler software (CAMO Technologies Inc. Woodbridge, N.J.).

The stability of the PCA models were tested using jack-knife cross-validation. This procedure is based on successively deleting one sample or a certain percentage of the observations from the data. The rest of the data are used for building the model. The model is then tested on the observations kept out of the computations and the predicted residual variance is computed. The procedure is repeated until all samples have been deleted once. Finally, the total residual variance is determined by averaging the individual contributions from each segment. The square root of the residual predictive variance is the root mean square error of prediction (RMSEP).

RFMCA Analyses

Five μl of the amplification products were digested using a restriction enzyme mixture (MspI, AluI, MseI and RsaI; 10 U each) in a total volume of 20 μl 1× NEB buffer 2 (New England BioLabs, Beverly, Mass.) at 37° C. for 8 hours followed by an enzyme inactivation at 65° C. for 5 min. The same approach was used for both the RFMCA and tRFLP samples.

For RFMCA, SYBR® Green I stain (Molecular Probes, Willow Creek, Oreg.) was added to the restriction enzyme cut reactions to a concentration of 10× in a total volume of 25 μl. The melting reactions were performed using the 7900HT system (Applied Biosystems). The SDS 2.2 software (Applied Biosystems) was used to analyse the data generated with the 7900 HT system.

Principal component analyses (PCA) and partial least square regression (PLSR) were used in combination with the prediction tools provided in the Unscrambler software in order to develop a classification model for the RFMCA data. The multimer frequency data were used as Y and spectral information as X in the classification model. The PCA and PLSR analyses were performed using full cross validation with centred data. The variables were weighted according to their standard deviations. The predictions were performed by first building a PLSR model using a calibration set, and then validating the model using an independent validation set of samples. The input data were normalized by subtracting the mean, and dividing by the standard deviation. The loading for the initial solution was computed from the data. The derived model was subsequently used for classification of new strains.

Database Storage and Retrieval

The RFMCA data were stored in a Microsoft Office Access™ database. The information about strain names, values for PC 1 and 2 and the maximum residual were included in the database. Standard SQL queries were used to retrieve information from the database, and for strain classification.

DNA Sequence Analyses

The sequence-characterized strains were subjected to a mega-BLAST search in the NCBI database (Altschul, S. F et al. 1990. J Mol Biol 215:403-10.). A relatively wide diversity of bacterial species were identified (Table 1). The AIBIMM analyses showed that there were four main groups of bacteria (Cluster I-IV, FIG. 2). Bacteria belonging to Clusters I, II and III were separated along the first principal component, while bacteria within Cluster N was separated along the second principal component. Cluster I contains bacteria belonging to the genus Streptococcus, while bacteria in Cluster III belong to the genus Staphylococcus. Clusters II and IV and contain several different genera. The main genera within cluster II are Carnobacterium and Bacillus. The bacteria within cluster IV belong to the Actinomycetales, represented by the genera Rothia, Actinomyces, Arthrobacter and Micrococcus.

The main structures in the data were that heat treated pepper was associated with Bacillus spp., and Staphylococcus spp. with curry chicken, while Streptococcus spp. and Actinomycetales were associated with finnbeef. The herb sauce contained a wide diversity of different bacterial groups (FIG. 2).

Restriction Cutting Site Information

The frequency distribution of the cutting sites in the sequences were analysed as theoretical evaluation of the discriminatory power of the restriction enzyme cutting. The restriction MspI and MseI were the most frequently occurring with mean frequencies of 2 and 1.7, respectively, within the 466 by fragment analysed. The restriction site AluI and RsaI had lower frequencies, and occurred respectively on average 1 and 0.9 times, respectively. PCA was used to evaluate the discriminatory power of the restriction site information. We were able to identify the same four clusters as for the DNA sequence analyses (results not shown). However, we were not able to differentiate the different strains within the clusters.

Discriminatory Power of RFMCA

The next step was to evaluate the discriminatory power of RFMCA analysis. A set of 26 strains was used develop a classification model for the RFMCA data, while 68 strains were used in the validation. Ten of the strains gave weak signals due to bad PCR amplification. The rest of the strains showed three major groups for the first principal component. These groups correspond to the clusters identified for the DNA sequence data. However, it was not possible to separate Cluster II from IV (FIG. 2A). These clusters could be separated in the second principal component for the RFMCA data (FIG. 2B). Characteristic RFMCA patterns for bacteria belonging to Clusters I to IV are shown in FIG. 3.

Database and Classification Rules

The bacterial strains were classified based on SQL query. For each sample the two variables with the highest residual after classification were identified. These values were included in the database, in addition to the predicted values for PC1 and PC2.

An empirical threshold of 0.5 for both variables with the highest residuals was determined. If both variables have higher values than 0.5, then the strain was not assigned to any of Clusters I to IV. The next criterion was to evaluate PC2. The strains were unlikely identified as belonging to Cluster IV if the value was above 9.5. The final separation was for Clusters I, II and III. The strains were assigned to cluster I if the PC1 scores were between −15 and −2, if the scores were between −2 and 5 then they were assigned to Cluster II, while for scores were between 5 and 15 then the strains were assigned to Cluster III.

The same classification for all the strains was obtained with both 16S rDNA sequence and RFMCA analyses (Table 1). The 10 strains with weak PCR amplification, however, were not assigned to any of Clusters I-IV. The reason is probably because it is the noise that is dominating the measurements and not the real phylogenetic signal. RFMCA for the bacterial species Eschericia coli, Campylobacter jejuni and Pseudomonas spp were also evaluated. All these bacteria were classified outside the model by the criterion of variable residuals (Table 1).

TABLE 1
Bacterial strains isolated and analysed

| Strain # | BLAST homology | 16S rDNA | RFMCA | Origin | Properties |
|---|---|---|---|---|---|
| 04-10-1 | Staphylococcus epidermidis | Cluster III | Cluster III | Herb sauce | aerobe sporeformer |
| 04-11-1 | Streptococcus sanguis | Cluster I | Cluster I | Herb sauce | aerobe sporeformer |
| 04-11-2 | Streptococcus mutans | Cluster I | missing | Herb sauce | aerobe sporeformer |
| 04-11-3 | neg | missing | Cluster I | Herb sauce | aerobe sporeformer |
| 04-11-4 | Streptococcus sanguis | Cluster I | Cluster I | Herb sauce | aerobe sporeformer |
| 04-11-5a | Streptococcus sanguis | Cluster I | Cluster I | Herb sauce | aerobe sporeformer |
| 04-11-5b | missing | missing | Cluster I | Herb sauce | aerobe sporeformer |
| 04-12-1 | Streptococcus salivarius | Cluster I | Cluster I | Herb sauce | aerobe sporeformer |
| 04-12-2 | Streptococcus mitis | Cluster I | Cluster I | Herb sauce | aerobe sporeformer |
| 04-12-3 | Streptococcus mitis | Cluster I | Cluster I | Herb sauce | aerobe sporeformer |
| 04-12-4 | Streptococcus mitis | Cluster I | Cluster I | Herb sauce | partial |
| | | | | | aerobe/predominant |
| | | | | | anaerobe |
| 04-13-1 | Streptococcus salivarius | Cluster I | Cluster I | Herb sauce | aerobe |
| 04-13-2 | Streptococcus salivarius | Cluster I | Cluster I | Herb sauce | aerobe |
| 04-13-3 | Streptococcus salivarius | Cluster I | missing | Herb sauce | aerobe |
| 04-13-5a | Streptococcus parasanguinis | Cluster I | Cluster I | Herb sauce | aerobe |
| 04-13-5b | missing | missing | Cluster I | Herb sauce | aerobe |
| 04-13-6a | Streptococcus parasanguinis | Cluster I | Cluster I | Herb sauce | aerobe |
| 04-13-6b | missing | missing | Cluster I | Herb sauce | aerobe |
| 04-13-7 | Streptococcus mitis | Cluster I | Cluster I | Herb sauce | aerobe |
| 04-13-8 | Streptococcus parasanguinis | Cluster I | Cluster I | Herb sauce | parrtial |
| | | | | | aerobe/predominant |
| | | | | | anaerobe |
| 04-15-1 | Staphylococcus pasteuri | Cluster III | Cluster III | Finnbeef | aerobe |
| 04-17-1 | Rothia sp. | Cluster IV | Cluster IV | Herb sauce | aerobe |
| 04-17-2 | Rothia sp. | Cluster IV | Cluster IV | Herb sauce | aerobe |
| 04-17-3 | Streptococcus salivarius | Cluster I | Cluster I | Herb sauce | aerobe |
| 04-17-4 | Staphylococcus pasteuri | Cluster III | Cluster III | Herb sauce | aerobe |
| 04-17-5 | Streptococcus sanguinis | Cluster I | Cluster I | Herb sauce | aerobe |
| 04-17-6 | Streptococcus sanguinis | Cluster I | Cluster I | Herb sauce | aerobe |
| 04-17-7 | neg | -missing | Cluster I | Herb sauce | aerobe |
| 04-17-8a | Streptococcus mitis | Cluster I | Cluster I | Herb sauce | aerobe |
| 04-17-8b | missing | missing | Cluster I | Herb sauce | aerobe |
| 04-17-9 | Streptococcus sanguinis | Cluster I | Cluster I | Herb sauce | aerobe |
| 04-17-10 | Streptococcus sanguinis | Cluster I | Cluster I | Herb sauce | aerobe |
| 04-17-11 | Streptococcus salivarius | Cluster I | Cluster I | Herb sauce | aerobe |
| 04-17-12 | Streptococcus sanguinis | Cluster I | Cluster I | Herb sauce | aerobe |
| 04-17-13 | Streptococcus salivarius | Cluster I | Cluster I | Herb sauce | aerobe |
| 04-18-1 | Staphylococcus pasteuri | Cluster III | Cluster III | Herb sauce | aerobe |
| 04-18-2 | neg | — | | Herb sauce | aerobe |
| 04-19-1 | Rothia sp | Cluster IV | Cluster IV | Herb sauce | aerobe |
| 04-20-1a | Staphylococcus hominis | Cluster III | Cluster III | Curry chicken | aerobe |
| 04-20-1b | missing | missing | Cluster III | Curry chicken | aerobe |
| 04-20-2a | Staphylococcus hominis | Cluster III | Cluster III | Curry chicken | aerobe |
| 04-20-2b | missing | missing | Cluster III | Curry chicken | aerobe |
| 04-20-3a | Staphylococcus hominis | Cluster III | Cluster III | Curry chicken | aerobe |
| 04-20-3b | missing | missing | Cluster III | Curry chicken | aerobe |
| 04-20-4a | Staphylococcus hominis | Cluster III | Cluster III | Curry chicken | aerobe |
| 04-20-4b | missing | missing | Cluster III | | |
| 04-21-2 | Micrococcus luteus | Cluster IV | Cluster IV | Finnbeef | aerobe |
| 04-22-2 | Pseudomonas putida | Cluster III | missing | Herb sauce | aerobe |
| 04-22-4 | Staphylococcus pasteuri | Cluster III | missing | Herb sauce | aerobe |
| 04-22-5 | Staphylococcus pasteuri | Cluster III | missing | Herb sauce | aerobe |
| 04-22-6 | Actinomyces naeslundii | Cluster IV | missing | Herb sauce | aerobe |
| 04-23-1 | Arthrobacter agilis | Cluster IV | missing | Herb sauce | aerobe |
| 04-23-2 | Staphylococcus epidermidis | Cluster III | missing | Herb sauce | aerobe |
| 04-25-1 | Arthrobacter sp. | Cluster IV | missing | Finnbeef | aerobe |
| 04-26-1 | Streptococcus salivarius | Cluster I | missing | Finnbeef | aerobe |
| 04-26-2 | Streptococcus sanguinis | Cluster I | missing | Finnbeef | aerobe |
| 04-26-3 | Actinomyces naeslundii | Cluster IV | missing | Finnbeef | aerobe |
| 04-26-7 | Staphylococcus epidermidis | Cluster III | Cluster III | Finnbeef | aerobe |
| 04-26-8 | Veillonella dispar | Cluster II | missing | Finnbeef | anaerobe |
| 04-27-1 | Streptococcus sanguinis | Cluster I | missing | Finnbeef | aerobe |
| 04-27-2 | Streptococcus sanguinis | Cluster I | missing | Finnbeef | aerobe |
| 04-27-3 | Streptococcus mitis | Cluster I | missing | Finnbeef | aerobe |
| 04-27-4 | Streptococcus mitis | Cluster I | missing | Finnbeef | aerobe |
| 04-28-2 | Bacillus subtilis | Cluster III | Cluster III | Varmebeh.sort | aerobe |
| | | | | pepper | |
| 04-28-4 | Bacillus subtilis | Cluster III | Cluster III | Varmebeh.sort | aerobe |
| | | | | pepper | |
| 04-28-5 | Bacillus pumilus | Cluster III | Cluster III | Varmebeh.sort | aerobe |
| | | | | pepper | |
| 04-28-6 | Bacillus subtilis | Cluster III | Cluster III | Varmebeh.sort | aerobe |
| | | | | pepper | |
| 04-28-7 | Bacillus subtilis | Cluster III | Cluster III | Varmebeh.sort | aerobe |
| | | | | pepper | |
| 04-28-8 | Bacillus clausii | Cluster III | Cluster III | Varmebeh.sort | aerobe |
| | | | | pepper | |
| 04-31-1 | missing | missing | Cluster II | Curry chicken | aerobe |
| 04-31-4 | Staphylococcus epidermidis | Cluster III | Cluster III | Curry chicken | aerobe |
| 04-32-4 | Brochothrix thermosphacta | Cluster III | missing | Herb sauce | aerobe |
| 04-32-5 | Carnobacterium divergens | Cluster II | Cluster II | Herb sauce | aerobe |
| 04-32-6 | Carnobacterium divergens | Cluster II | Cluster II | Herb sauce | aerobe |
| 04-32-7 | Carnobacterium divergens | Cluster II | Cluster II | Herb sauce | aerobe |

Application of RFMCA for Quality Control

Different product categories are often associated with distinct groups of microorganisms. Classification models can thus be made for the microorganisms expected in a given product. Such models can subsequently be used for high throughput classification. If microorganisms are detected that are outside the groups for which the model was built, then these can be classified by 16S rDNA sequencing. These microorganisms can also be included in the RFMCA model for future rapid classification. Databases with information about a given product, or category of products can in this way be developed.

### EXAMPLE 2

DNA Purification from Cecal Samples

Cecal samples from two chicken flocks raised in the eastern part of Norway in August 2003 were used for the optimisation and the evaluation of the robustness of the RFMCA method. The flocks were raised by two different producers (abbreviated W and M) under similar conditions (in standard broiler houses) and feeding regimes (Felleskjøpet AS, Oslo, Norway).

Immediately after slaughter, the ceca were transported on ice to the test laboratory, and stored at −40° C. After thawing, 50 mg/ml cecum content was suspended in 4 M guanidine thiocyanate (GTC). Two-fold dilution series (0, 1:2, 1:4, and 1:8) in 4M GTC were made and each dilution was processed in duplicate by transferring 500 μl to sterile FastPrep®-tubes (Qbiogene Inc, Carlsbad, Calif.) containing 250 mg glass beads (106 microns and finer, Sigma, Steinheim, Germany). The samples were homogenized for 80 s in a FastPrep® Instrument (QBiogene). DNA purification was done using MagPrep® silica particles (Merck, Darmstadt, Germany) following the manufacturer's recommendations in a Biomek® 2000 Workstation (Beckman Coulter, Fullerton, Calif.) (Skånseng, B, and K. Rudi. 2004. AFAC workshop, Alternatives to feed antibiotics and anticoccidials in the pig and poultry meat production, 19-20 Sep. 2004, Århus, Denmark.).

PCR Amplification

16S rRNA gene sequences were amplified using universal primers 5′TCC TAC GGG AGG CAG CAG T3′ (forward) and 5′GGA CTA CCA GGG TAT CTA TTC CTG TT3′ (reverse). The primers amplify the region from 331 to 797 in the Escherichia coli 16S rRNA sequence (Nadkarni, M. A., et al. 2002. Microbiology 148:257-266.). The forward primer was labelled with 6-FAM and the reverse primer labelled with TAMRA for the tRFLP analyses, while unlabelled primers were used for DNA sequencing and RFMCA.

The 25 ml reactions contained 1× AmpliTaq Gold reaction buffer (Applied Biosystems, Foster City, Calif.), 1 mM MgCl₂, 1 mM dNTP's, 1 μM of each primer, and 1 U AmpliTaq Gold DNA polymerase (Applied Biosystems). The amplification profile used was as follows: 95° C. for 30 s, 65° C. for 30 s, and 72° C. for 45 s for 35 cycles. The enzyme was activated and target DNA denatured at 10 min for 95° C. prior to amplification, and an extension step of 7 min at 72° C. was included after the amplification. The reactions were performed using a GeneAmp PCR System 9700 (Applied Biosystems).

Cloning and DNA Sequencing

The TOPO TA Cloning® kit (Invitrogen, Carlsbad, Calif.) with TOP 10 One Shot® chemically competent cells was used for cloning. Transformation of the cells was performed as described in the TOPO TA Cloning manual. The Rapid One Shot® Chemical transformation protocol was used (Invitrogen). Plasmids from the positive colonies were isolated by re-suspending a colony in 30 μl water, heating to 99° C. for 5 min, removing the cell debris by centrifugation at 13 000 rpm (Biofuge Fresco, Kendro Laboratory Products, Asheville, N.C.) for 1 min, and transferring 25 ml to a new tube. The insert was amplified with the 5′-CGC CAG GGT TTT CCC AGT CAC GAC G-3′ (HU) and 5′-GCT TCC GGC TCG TAT GTT GTG TGG-3′ (HR) primers, which are specific for the vector. The following amplification reaction was used: 95° C. for 4 min and then 95° C. for 15 s, 65° C. for 30 s, and 72° C. for 1 min for 30 cycles. The reaction was ended with an extension step at 72° C. for 7 min.

The presequencing reaction included treating 8 μl of the PCR product with 10 U exonuclease I (Amersham, Piscataway, N.J.) and 2 U shrimp alkaline phosphatase (Amersham) at 37° C. for 15 min. The enzymes were inactivated by heating to 80° C. for 15 min. Sequencing was done using the Big Dye™ Terminator v 2.0 Cycle Sequencing Kit (Applied Biosystems) on an ABI Prism 3100 Genetic Analyzer (Applied Biosystems). Preparation of the sequencing mixture was performed as recommended by the manufacturer.

Restriction Enzyme Digestion

Five μl of each of the amplification products was digested using a restriction enzyme mixture (MspI, AluI, MseI and RsaI; 10 U each) in a total volume of 20 μl 1× NEB buffer 2 (New England BioLabs, Beverly, Mass.) at 37° C. for 8 hours followed by an enzyme inactivation at 65° C. for 5 min. The same approach was used for both the RFMCA and tRFLP samples.

RFMCA Melting

For RFMCA, SYBR® Green I stain 10 000× stock solution (Molecular Probes, Willow Creek, Oreg.) was added to the restriction enzyme cut reactions to a concentration of 10× in a total volume of 25 μl. The melting reactions were performed using either an ABI Prism 7700 Sequence Detection System or the 7900HT system (Applied Biosystems). Dissociation Curves 1.0 software (Applied Biosystems) was used to analyse the melting patterns for the 7700 data, while SDS 2.2 software (Applied Biosystems) were used to analyse the data generated with the 7900 HT system.

tRFLP Size Separation

The tRFLP samples were separated in a 3% agarose gel at 100 volts for 1 hour. The detection was done using a Typhoon 8600 Variable Mode Imager (Amersham). Quantification was performed using ImageMaster Total Lab software (Amersham).

Phylogenetic Reconstruction and Cluster Analyses

Sequences of representative strains were selected from the Genbank nucleotide sequence database (March, 2004) based on searches with the BLAST program (www.ncbi.gov) and aligned with sequences obtained in this study using Clustal X (Thompson, J D., et al. 1997. Nucl Acids Res 25: 4876-4882). The alignments were then manually edited using the program BioEdit (Hall, T A. 1999. Nucl Acids Symp Ser 41: 95-98). A phylogenetic tree was constructed using Tamura Nei distances (Tamura, K., and M. Nei. 1993. Mol Biol Evol 10:512-526) and the Minimum Evolution algorithm provided in the MEGA 2 software-package (Kumar, S., K. et al. 2001. Bioinformatics 17:1244-1245.). Statistical support for the branches in these trees was obtained by bootstrap analysis with 500 replicates.

The RFMCA data were clustered using correlation coefficient distances, and Ward linkage for dendrogram construction (Minitab v. 14, Minitab Inc, State College, Pa.). The RFMCA input data were normalized by subtracting the mean, and dividing by the standard deviation for each data-point, prior to the cluster analyses.

Statistical Analyses

Two tail t-tests and tests for standard deviation provided in the Minitab v. 14 software package (Minitab Inc, State College, Pa.) were used. The multivariate statistical analyses were performed using The Unscrambler® v. 9.0 software (Camo Inc, Woodbridge, N.J.). Principal component analyses (PCA) and partial least square regression (PLSR) in combination with the prediction tools provided in the Unscrambler software were used. The PCA and PLSR analyses were performed using full cross validation with centred data. The variables were weighted according to their standard deviations. The prediction was performed by first building a PLSR model using a calibration set. The model was then validated using an independent sample set. The input data were normalized by subtracting the mean and dividing by the standard deviation. The loading for the initial solution was computed from the data.

Optimising the Resolution of RFMCA

The parameters tested were restriction enzyme combinations, melting temperature range, and stringency. The results are summarized in Table 2.

The restriction enzymes used for RFMCA should be compatible with the same buffer system and frequent cutters. The four restriction enzymes MspI (C▾CGG), AluI, (AG▾CT), MseI (T▾TAA) and RsaI (GT▾AC) meet these criteria. These enzymes were used in the optimisation of the RFMCA method. The resolution for samples cut with single enzymes was lower than the samples cut with all four enzymes. The theoretical average fragment size of 256 bp for the samples cut by single enzymes is probably too large to be separated by melting point analyses. The theoretical average size of the fragments for the combination of the four enzymes is 64 bp which is probably within the range that can be separated by melting point analysis.

The greatest levels differentiation and reproducibility within melting peak boundaries of ±2.5° C. was obtained in the melting temperatures range of 65-92° C. (see FIG. 1A for typical pattern). The melting patterns obtained below 65° C. were relatively unstable, possibly due to variable accumulation of small fragments such as primer dimers. All the fragments were melted above 92° C., and thus no useful information was obtained above that temperature.

It was then assessed whether modifying the stringency of the reaction could increase the resolution of RFMCA. The stringency of the reaction was lowered by the addition of high salt standard saline citrate (SSC) solution, while the stringency of the reaction was increased by adding the cosolvent dimethylsulfoxide (DMSO).

Both SSC and DMSO led to less distinct melting peak patterns and lowered resolution (Table 2). It was concluded that SSC and DMSO did not improve the performance of RFMCA. These compounds were therefore not used further.

The final, optimised, RFMCA protocol involved cutting with all four restriction enzymes and melting in the range 65-95° C. for 20 min, while only data for the temperature range of 65-92° C. were used for the subsequent discrimination analyses.

TABLE 2
Evaluation and optimization of RFMCA parameters¹

| Parameters | Conditions tested | Optimum | Comments |
|---|---|---|---|
| Temperature | 4-95 | 65-92 | Irreproducible signals |
| (° C.) | | | below 65° C. and all |
| | | | fragments were melted |
| | | | above 92° C. |
| DSMO (%) | 0, 0.5, 1, 3 | 0 | DSMO gave diffuse peak |
| | | | patterns |
| SSC (×) | 0, 0.5, 1, 10 | 0 | SSC gave diffuse peak |
| | | | patterns |
| Restriction | AluI, MspI, MseI, | All | Combination of all four |
| enzymes | and RsaI | enzymes | enzymes gave the best |
| | | | resolution |

¹The optimization was done on a random set of 6 DNA segments cloned from cecal samples. The analyses were run in triplicate.

Application of RFMCA for Characterisation of Complex Communities in Chicken Cecal Samples

The reproducibility and discriminatory power of RFMCA were evaluated by in-depth comparisons of the two closely related microbial communities W and M (see Materials and Methods for details). An initial characterisation of the diversity in the samples was performed by cloning and sequencing of partial 16S rRNA gene sequences. The cloned fragments were subsequently subjected to RFMCA. Three major RFMCA patterns (A to C) were identified from these clones using correlation coefficient distances and Ward linkage for dendrogram construction (FIG. 5B).

There was a good correspondence between RFMCA and DNA sequence classification (results not shown). Basically, RFMCA pattern A corresponded to Clostridiales, B corresponded to Bacteroidales, while C corresponded to Bacillales, Lactobacillales and uncultured gram-positive bacteria.

The RFMCA principle was further evaluated by direct analyses of the microbial communities in the cecal content from the W and M samples. Eight independent DNA purifications consisting of duplicate analyses of each of the dilutions (0, 1:2, 1:4, and 1:8) described in Materials and Methods were analysed for each of the samples (FIG. 6A). Diagnostic peaks for the A groups of bacteria were identified in the W sample, while there were peaks corresponding to the C group of bacteria in the M sample (see arrows in FIG. 6A). Clear differences in the microbial communities using principal component analyses could also be detected. The first principal component gave an average score of 1.82±0.71 for W and −2.64±0.38 for M, respectively. These scores were found significantly different using a two tail t-test (T=15.37 and P<0.0005).

A theoretical evaluation of the expected restriction fragments identified by tRFLP was performed. Fragments of 146 and 124 bp for clones belonging to cluster C were identified, while the expected fragments for clones belonging to cluster A were 87 and 72 bp. Two tRFLP bands that were discriminatory between the W and M samples (T=4.87 and P=0.001; FIG. 6B) were identified, which probably correspond to the theoretically identified 146 and 124 bp and the 87 and 72 bp fragments, respectively. A resolution of approximately ±10 bp was determined for our tRFLP by comparison with known molecular weight standards (results not shown).

Evaluation of RFMCA for Defined Samples.

Representative samples with restriction digestion patterns resembling pattern A, B and C were chosen for evaluating the performance of RFMCA and tRFLP (FIG. 7). The samples were mixed according to the experimental design shown in FIG. 7A. Regression models were first built using a calibration set of data. The accuracy of these models were then evaluated using a new set of independent validation data (FIG. 7B). These analyses showed that RFMCA overall gave a good accuracy and precision (FIG. 7B). The misclassification for the RFMCA data was <15%. This example also shows that it should be possible to quantify the composition of mixed bacterial populations if the patterns for the pure components are known. Such an application would be particularly important in process or quality control where known mixtures of bacteria are used, such as in e.g. food fermentation. The reason for the relatively high error rate for the tRFLP data, however, may be due to relatively low resolution of the agarose gel electrophoresis applied. Our tRFLP results may not be representative for other separation techniques such as high-throughput capillary gel electrophoresis.

1. A method of classifying a microorganism present in a sample comprising the steps of:
a) digesting nucleic acid derived from the microorganism with at least one restriction enzyme; and
b) determining the melting profile of the restriction fragments produced in step a).

2. The method of claim 1 wherein a target region in the nucleic acid of the microorganism is amplified prior to step (a).

3. The method of claim 2 wherein said target region is a region of nucleic acid in which evolutionary differences between different taxonomic families and/or genera and/or species are present in the sequence of the target region.

4. The method of either claim 2 wherein said target region is 16S DNA.

5. The method of claim 2 wherein said target region is the spacer between 16S and 23S DNA.

6. The method of claim 1 wherein the microorganism is classified to the level of taxonomic family.

7. The method of claim 1, wherein the microorganism is classified to the level of taxonomic genus.

8. The method of claim 1, wherein the microorganism is classified to the level of taxonomic species.

9. The method of claim 1, wherein at least three, preferably at least four restriction enzymes are used to digest the nucleic acid.

10. The method of claim 1, wherein at least 5 different fragments, preferably at least 10 different fragments are obtained through step (a).

11. The method of claim 1, wherein 10 to 30 different fragments are obtained through step (a).

12. The method of claim 1, wherein the length of said fragments is between 300 and 30 bp, preferably between 100 and 50 bp.

13. The method of claim 1, wherein the melting point range of said fragments is 65-92° C.

14. The method of claim 1, wherein the at least one restriction enzyme is selected from the group consisting of Msp1, AluI, MseI, RsaI and combinations thereof.

15. The method of claim 1, wherein all steps are performed in a single vessel.

16. The method of claim 1, further comprising a step (c) of comparing the melting profile obtained in step (b) with said at least one reference melting profile.

17. The method of claim 1, wherein the melting profile obtained in step (b) is analysed using bilinear modelling or multivariate regression methods.

18. The method of claim 17 wherein said bilinear modelling method is principal component analyses and the multivariate regression method is partial least square regression.

19. The method of claim 17 further comprising a step wherein the analysis results are clustered around a predetermined phylogenetic tree to provide clustering information.

20. The method of claim 19 wherein said phylogenetic tree has been predetermined using nucleic acid sequencing techniques.

21. The method of either claim 19 wherein said clustering information is obtained using correlation coefficient distances and Ward linkage for dendrogram construction.

22. The method of claim 19, further comprising a step wherein the clustering information obtained is compared with at least one reference set of clustering information.

23. The method of claim 16 wherein the reference melting profile is retrieved from a database stored on a data processing system.

24. The method of claim 22 wherein the reference set of clustering information is retrieved from a database stored on a data processing system.

25. The method of claim 1, further including the step of storing the resulting melting profile on a database.

26. The method of claim 19, further including the step of storing the resulting clustering information on a database.

27. A database stored on digital storage media, comprising melting profiles obtained by carrying out the method of claim 1.

28. A database stored on digital storage media, comprising clustering information obtained by carrying out the method of claim 1.

29. A method of classifying a cell from a higher eukaryote present in a sample comprising the steps of:
a) digesting nucleic acid derived from the microorganism with at least one restriction enzyme; and
b) determining the melting profile of the restriction fragments produced in step a).

30. The method of claim 29 wherein said target region is 16S DNA or the spacer between 16S and 23S DNA.

31. The method of claim 29 wherein at least three, preferably at least four restriction enzymes are used to digest the nucleic acid.

32. The method of claim 29, wherein at least 5 different fragments, preferably at least 10 different fragments are obtained through step (a).

33. The method of claim 29, wherein 10 to 30 different fragments are obtained through step (a).

34. A kit for use in a classification method of claim 1, said kit comprising:
(a) one or more restriction enzymes; optionally
(b) one or more primers suitable for performing an amplification reaction; optionally
(c) a restriction buffer; optionally
(d) a melting buffer; and optionally
(e) means for providing an indication of nucleic acid duplex dissociation.
